(19) United States
(12) Patent Application Publication
(10) Pub. No.: US 2012/0095348 A1
(43) Pub. Date: Apr. 19, 2012

(54) Three Dimensional Imaging Intra Cardiac Echocardiography (ICE) Catheter

(75) Inventor: Reinhard J. Warnking, Palm Beach Gardens, FL (US)

(73) Assignee: Sonavation, Inc., Palm Beach Gardens, FL (US)

(21) Appl. No.: 13/277,039

(22) Filed: Oct. 19, 2011

Related U.S. Application Data
(60) Provisional application No. 61/394,575, filed on Oct. 19, 2010.

(51) Int. Cl. A61B 8/14

(52) U.S. Cl. 600/459

(57) ABSTRACT

Provided herein is an ultrasound imaging device including a two dimensional ultrasound imaging array for three dimensional images positioned at least in the vicinity of distal end of a catheter configured to be used for intra organ imaging, wherein the imaging array includes a first number of output ports. The imaging device also includes a high voltage multiplexer configured to (i) reduce the first number of signal lines to a second number of signal lines, wherein the second number is less than the first number and (ii) connect the array to a signal processing system.

### CROSS-REFERENCE TO RELATED APPLICATIONS

This application claims benefit to U.S. Provisional Application No. 61/394,575, filed on Oct. 19, 2010, which is incorporated herein by reference.

### BACKGROUND OF THE INVENTION

1. Field of the Invention

The present invention relates to ultrasound imaging apparatuses placed within an organ of a human subject to provide images of such organ.

2. Background Art

Both electronic as well as mechanical approaches to ICE imaging require significant training since the image represents just a 2 dimensional cross section through the heart which makes catheter positioning critical (see Advances in Catheter Based Ultrasound Imaging, T. L. Proulx . . . 2005 IEEE Ultrasonics Symposium). Therefore approaches have been proposed to improve catheter steering, see US 2008/0009745 A1 by Hossak. Another way to facilitate easier catheter positioning and easier image interpretation is proposed in U.S. Pat. No. 5,876,345 Eaton by combining 2 arrays at the distal catheter end.

In the US and many other countries heart disease is a leading cause of death and disability. One kind of heart disease is Atrial Fibrillation (AF) which leads to an uncoordinated, diminished pumping function of the left atrium and a significantly increased risk of stroke.

The medical profession utilizes a wide variety of tools to treat heart disease and in particular AF ranging from drugs to open heart surgery. Often a minimally invasive catheter based procedure will be used to treat the heart disease. In the case of AF these procedures are catheter based ablation procedures where certain lesions (tissue necrosis) are generated in the left atrium (typically circular lesions surrounding the pulmonary vein ostiae) to create electrical conduction blocks in order to interrupt unwanted electrical pathways responsible for the AF. The ablation procedure as well as other catheter based heart procedures are often guided by intra cardiac echocardiography or ICE. ICE guidance has the advantage that real time imaging with morphological information is obtained without any dangerous radiation burden (fluro guidance does not provide morphological information).

All left sided atrial interventions require a transeptal puncture to be performed. Here ICE guidance has great value since tenting of the septum indicates clearly the location of the puncture site.

There are two types of ICE technologies; a mechanically rotating transducer mounted on a drive shaft within the lumen of a catheter and the full electronic phased array approach. The mechanical approach generates a radial or circumferential image or field of view. Since the transducer aperture is limited by the catheter diameter (which is typically in the 7 F range) penetration is inferior to the penetration of a phased array which utilizes a larger aperture extending in the axial catheter direction. Therefore the mechanical ICE catheter needs to be placed close to the site of the interest, in the case of AF at the ablation site. Typically, with a mechanical ICE catheter, an imaging run is performed prior to intervention, then, the ICE catheter is being withdrawn and exchanged for the therapy catheter. This exchange between imaging and therapeutic catheter is being repeated depending on the complexity of the case. Obviously it is cumbersome and the disadvantage is that the operator does not obtain real time guidance for the therapeutic action unless a separate transeptal puncture only for the imaging catheter is being performed.

The electronic side looking phased array type catheter is positioned in the right atrium and therefore does provide real time guidance for the therapeutic action, tissue ablation, in the case of an AF procedure. However, since the imaging catheter is located in the right atrium, relatively far away from the therapeutic action (in case of an AF procedure 5 to 10 cm for the left pulmonary vein isolation sites) catheter orientation is critical. The slightest rotation of the ICE catheter will change the imaging site completely. Both the electronic as well as the mechanical approach to ICE imaging require intense training Since the image represents just a two dimensional cross section through the heart, catheter positioning is very critical and requires significant experience. Therefore a need exists for easier to position and interpret ICE images like three dimensional images which reduce the need for continuous catheter manipulation and make the images easier to interpret.

Embodiments of the present invention also include an apparatus for guiding catheter ablation.

What are needed, therefore, are methods and systems that support an all electronic three dimensional disposable imaging catheter.

### BRIEF SUMMARY OF EMBODIMENTS OF THE INVENTION

The present invention meets the above-described needs by providing a true all electronic three dimensional disposable imaging catheter unlike array combinations as proposed by Eaton or mechanically rotated arrays (see Van der Steen, Erasmus University). A disposable true 3 dimensional ICE catheter is possible by utilizing a low cost, flexible ultrasound array technology, as described in U.S. patent application entitled Method for Making a Piezo Ceramic Body, Application No. 61/218,690, incorporated herein by reference. The cost factor allows for disposable applications on a catheter.

The imaging information is complemented by color flow mapping to highlight blood—vessels and—chambers.

Embodiments of the present invention utilize ultrasound array technology to construct a two dimensional miniature array located in the distal tip of an ICE catheter combined with highly integrated multiplexer IC's.

These embodiments relate more specifically to Intra Cardiac Echocardiography (ICE) apparatuses as described in U.S. Pat. No. 4,917,097 Proudian; U.S. Pat. No. 5,857,974 Eberle; U.S. Pat. No. 5,795,299 Eaton and Seward, J. B.; D. L. Packer “Ultrasound Cardioscopy: Embarking on a new Journey” Mayo Clinic Proceedings, 71(7):629-635.

Further features and advantages of the invention, as well as the structure and operation of various embodiments of the invention, are described in detail below with reference to the accompanying drawings. It is noted that the invention is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the present invention and, together with the description, further serve to explain the principles of the invention and to enable a person skilled in the relevant art(s) to make and use the invention.

FIG. 1 is an exemplary illustration of a two dimensional ultrasound imaging array constructed in accordance with embodiments of the present invention.

The features and advantages of the present invention will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF EMBODIMENTS OF THE INVENTION

In the detailed description that follows, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

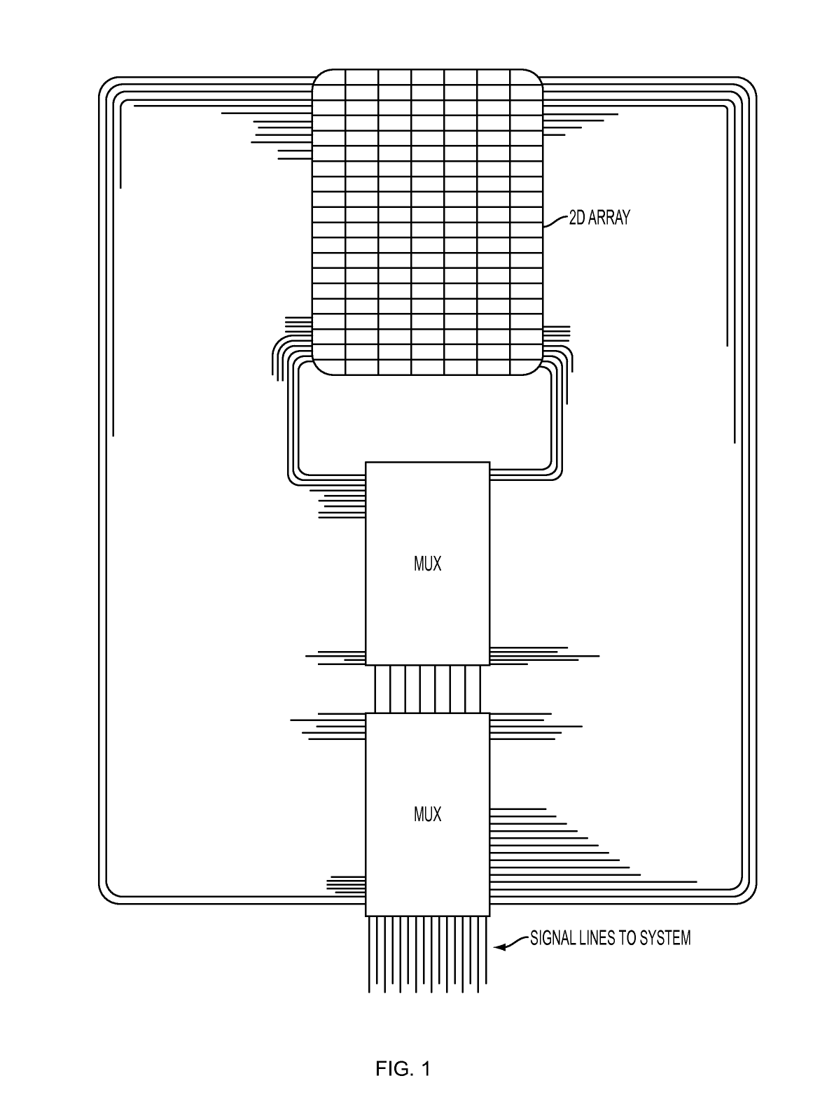

FIG. 1 is an exemplary illustration of a two dimensional ultrasound imaging array constructed in accordance with embodiments of the present invention.

Real time 3 dimensional catheter imaging requires miniature two dimensional arrays operating in the 7 to 10 MHz range. To build such arrays with conventional techniques would be prohibitively expensive and commercially not feasible since these imaging catheters are disposable products. The array manufacturing process, as described in the patent application above, allows for low cost production to meet the cost targets for a disposable product.

An array including several hundred elements will not allow for conventional techniques to connect each element via coax cables or flex circuits (strip lines) with the beam-former of an imaging system due to space limitations in the catheter shaft (typically 7 to 12 F). Therefore, highly integrated multiplexer integrated circuits (IC's) have been developed to reduce the number of signal lines from several hundred to 16 or 32. Embodiments of the present invention provide cost effective 3D disposable catheters, with the features described below.

Catheters in accordance with the present invention can be disposable. To manufacture a 2D array can cost about 1K, which prevents it from being disposable. Any suitable matrix array can be used. Such an array can be a 64×64 array, or any other suitable configuration. The array can reasonably be manufactured using the molding and manufacturing process noted above.

Additionally, catheters designed in accordance with the present invention can be deposited on the end of a probe or the distal tip of an ice catheter (sheath, and glove). Alternatively, the catheters can be deposited on the distal tip of a steerable deflectable catheter.

Next, electronic switches, such as multiplexers, can be used to run all of the related cables (64×64) through a thin catheter which can be about 3 mm diameter max. Switches, such as multiplexers, can be used to reduce the number of cables to a manageable number. In the embodiments, such a multiplexer facilitates close placement of the array to the sensor.

Groups of elements can be handled at a time. For example, groups of 16 can be used because these can be multiplexed down to 16 signal lines which go to the imaging system console display. After this group of 16, then the process steps over to another group of 16. The image in the system is then digitally constructed based upon the output from these lines.

### CONCLUSION

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

What is claimed is:

1. An ultrasound imaging device, comprising:
a two dimensional ultrasound imaging array positioned at least in the vicinity of distal end of a catheter configured to be used for intra organ imaging; and
a contact, non linear backing configured for attachment to the array to reduce array thickness.

2. An ultrasound imaging device, comprising:
a two dimensional ultrasound imaging array for three dimensional images positioned at least in the vicinity of distal end of a catheter configured to be used for intra organ imaging;
wherein the imaging array includes a first number of output ports; and
a high voltage multiplexer proximate to the array and configured to (i) reduce the first number of signal lines to a second number of signal lines, wherein the second number is less than the first number and (ii) connect the array to a signal processing system.

3. The ultrasound imaging device of claim 2, wherein the second number is an order of magnitude less than the first number.
